(19) United States
(12) Patent Application Publication
(10) Pub. No.: US 2019/0210996 A1
(43) Pub. Date: Jul. 11, 2019

(54) NOVEL COMPOUNDS

(71) Applicant: GLAXOSMITHKLINE INTELLECTUAL PROPERTY DEVELOPMENT LIMITED, Brentford, Middlesex (GB)

(72) Inventors: John David HARLING, Hertfordshire (GB); Christopher TINWORTH, Hertfordshire (GB)

(21) Appl. No.: 16/326,234

(22) PCT Filed: Aug. 16, 2017
(86) PCT No.: PCT/EP2017/070718

§ 371 Date: Feb. 18, 2019

(30) Foreign Application Priority Data
Aug. 18, 2016 (GB) 1614134.3

(51) Int. Cl. C07D 401/14; A61K 47/64; A61K 47/66; A61K 45/06

(52) U.S. Cl. CPC C07D 401/14; A61K 47/64; A61P 35/00; A61K 45/06; A61K 47/66

(57) ABSTRACT

A method of treating disorders associated with aberrant kinase activity, wherein the kinase is. Adaptor-associated protein kinase 1 (AAK1), Aurora Kinase A (AURKA), Aurora Kinase B (AURKB), Bruton's Tyrosine Kinase (BTK), Interleukin-1 receptor-associated kinase 3 (IRAK3), Protein tyrosine kinase 2 beta (PTK2B), Tyrosine-protein kinase Tec (TEC), Serine/threonine-protein kinase Wee1 (WEE1), Cyclin G-associated kinase (GAK), Large Tumour suppressor 1 Kinase (LATS1), Focal Adhesion Kinase (PTK2), Ribosomal protein S6 kinase alpha-1 (RPS6KA1) said method comprising degrading said kinase.

### FIELD OF THE INVENTION

The present invention relates to compounds, compositions, combinations and medicaments containing said compounds and processes for their preparation. The invention also relates to the use of said compounds, combinations, compositions and medicaments, for example as inhibitors of the activity of target proteins, including degrading target proteins and the treatment of disorders mediated by the target proteins.

### BACKGROUND OF THE INVENTION

An important large family of enzymes is the protein kinase enzyme family. Currently, there are about 500 different known protein kinases. Protein kinases serve to catalyze the phosphorylation of an amino acid side chain in various proteins by the transfer of the γ-phosphate of the ATP-Mg²⁺ complex to said amino acid side chain. These enzymes control the majority of the signaling processes inside cells, thereby governing cell function, growth, differentiation and destruction (apoptosis) through reversible phosphorylation of the hydroxyl groups of serine, threonine and tyrosine residues in proteins. Studies have shown that protein kinases are key regulators of many cell functions, including signal transduction, transcriptional regulation, cell motility, and cell division. Several oncogenes have also been shown to encode protein kinases, suggesting that kinases play a role in oncogenesis. These processes are highly regulated, often by complex intermeshed pathways where each kinase will itself be regulated by one or more kinases. Consequently, aberrant or inappropriate protein kinase activity can contribute to the rise of disease states associated with such aberrant kinase activity. Due to their physiological relevance, variety and ubiquitousness, protein kinases have become one of the most important and widely studied family of enzymes in biochemical and medical research.

The protein kinase family of enzymes is typically classified into two main subfamilies: Protein Tyrosine Kinases (PTK) and Protein Serine/Threonine Kinases, based on the amino acid residue they phosphorylate. The serine/threonine kinases (PSTK), includes cyclic AMP- and cyclic GMP-dependent protein kinases, calcium- and phospholipid-dependent protein kinase, calcium- and calmodulin-dependent protein kinases, casein kinases, cell division cycle protein kinases and others. These kinases are usually cytoplasmic or associated with the particulate fractions of cells, possibly by anchoring proteins. Aberrant protein serine/threonine kinase activity has been implicated or is suspected in a number of pathologies such as rheumatoid arthritis, psoriasis, septic shock, bone loss, many cancers and other proliferative diseases. Accordingly, serine/threonine kinases and the signal transduction pathways which they are part of are important targets for drug design. The tyrosine kinases phosphorylate tyrosine residues. Tyrosine kinases play an equally important role in cell regulation. These kinases include several receptors for molecules such as growth factors and hormones, including epidermal growth factor receptor, insulin receptor, platelet derived growth factor receptor and others. Studies have indicated that many tyrosine kinases are transmembrane proteins with their receptor domains located on the outside of the cell and their kinase domains on the inside. Much work is also under progress to identify kinase modulators.

It is desirable to identify inhibitors of kinase activity as potential therapies of disorders associated with aberrant kinase activity.

The selective degradation of target proteins using small molecules is a new approach to the treatment of various diseases. Proteolysis Targeting Chimeric molecules (Protacs) are bifunctional molecules which can simultaneously bind a target protein and an E3 ubiquitin ligase thereby bringing the ligase and target in close proximity These bifunctional molecules allow the efficient ubiquitin transfer from the ligase complex to the target protein which is subsequently recognized by the proteasome and degraded. This degradation of the target protein provides treatment of diseases or conditions modulated through the target protein by effectively lowering the level of said target protein in the cells of the patient. An advantage of Protacs is that a broad range of pharmacological activities is possible, consistent with the degradation/inhibition of targeted proteins from virtually any class or family.

E3 ubiquitin ligases (of which hundreds are known in humans) confer substrate specificity for ubiquitination and therefore are more attractive therapeutic targets than general proteasome inhibitors due to their specificity for certain protein substrates. The development of ligands for E3 ligases has proven challenging.

Thalidomide was first used in a clinical setting almost 60 years ago but only recently has its mechanism of action been more fully characterised with elegant work showing its primary target is cereblon, a part of the CRL4 E3 RING Cullin ligase complex (J. B. Bartlett, K. Dredge and A. G. Dalgleish, Nat. Rev. Cancer, 2004, 4, 314-322). Upon binding to cereblon, thalidomide and its analogues pomalidomide and lenalidomide (collectively known as IMiDs: immunomodulatory drugs,) create a neomorphic surface allowing recruitment, ubiquitination and subsequent degradation of transcription factors Ikaros and Aiolos. This results in IL-2 secretion and stimulation of T cells, and through this mechanism IMiDs demonstrate clinical efficacy in multiple myeloma.

Protacs employed to recruit target proteins to the E3 ligase cereblon have therefore been proposed, see for example WO2015/160845.

The present inventors have identified kinase targets which are capable of being degraded by Protacs comprising moieties that bind to cereblon as the E3 ligase, in particular the targets Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC).

### SUMMARY OF THE INVENTION

In one aspect of the present invention there is provided a method of treating disorders associated with aberrant kinase activity, wherein the kinase is Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC), said method comprising degrading said kinase.

In a further aspect of the present invention there is provided a method of degrading target proteins selected from Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC), by constructing Protac compounds or pharmaceutically acceptable salts thereof comprising E3 ligase binding moieties and target protein binding moieties linked directly or via a linking moiety, thus recruiting the target proteins to the E3 ligase allowing ubiquitin transfer from the ligase to the target protein enabling it to be recognized by the proteasome and degraded.

In a further aspect of the present invention there is provided a Protac compound or pharmaceutically acceptable salt thereof comprising moieties which binds to cereblon and a moiety which binds to a target protein selected from Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC) linked directly or via a linking moiety.

In one aspect there is provided a compound of Formula (I);

Target Protein Binder-Linker-cereblon binder (I)

or a pharmaceutically acceptable salt thereof wherein the target protein is Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC).

In a further aspect of the present invention, there is provided a compound of formula (I) or a pharmaceutically acceptable salt thereof for use in therapy.

In a further aspect there is provided a compound of formula (I) or a pharmaceutically acceptable salt thereof for use in the treatment of disorders mediated by the target protein.

In a further aspect of the present invention, there is provided a pharmaceutical composition comprising a compound of formula (I) or a pharmaceutically acceptable salt thereof and one or more of pharmaceutically acceptable carriers, diluents and excipients.

In a further aspect of the present invention, there is provided a method of treating disorders mediated by the target protein in a subject comprising administering a therapeutically effective amount of a compound of formula (I) or a pharmaceutically acceptable salt thereof.

In a further aspect of the present invention, there is provided the use of a compound of formula (I), or a pharmaceutically acceptable salt thereof in the manufacture of a medicament for use in treating disorders mediated by the target protein.

In a further aspect there is provided a combination comprising a compound of formula (I), or a pharmaceutically acceptable salt thereof and at least one further therapeutic agent.

In a further aspect there is provided a combination comprising a compound of formula (I) or a pharmaceutically acceptable salt thereof and at least one further therapeutic agent for use in therapy.

In a further aspect of the present invention, there is provided a pharmaceutical composition comprising a combination comprising a compound of formula (I) or a pharmaceutically acceptable salt thereof and at least one further therapeutic agent and one or more of pharmaceutically acceptable carriers, diluents and excipients.

In a further aspect of the invention there is provided a combination comprising compound of formula (I) or a pharmaceutically acceptable salt thereof and at least one further therapeutic agent for use in treating disorders mediated by the target protein.

In a further aspect there is provided a method of treating disorders mediated by the target protein comprising administering to a human in need thereof a therapeutically effective amount of a combination comprising compound of formula (I) or a pharmaceutically acceptable salt thereof, and at least one further therapeutic agent.

In a further aspect there is provided the use of a combination comprising compound of formula (I) or a pharmaceutically acceptable salt thereof and at least one further therapeutic agent in the manufacture of a medicament for treating disorders mediated by the target protein.

In a further aspect there is provided a method of degrading the target protein comprising administering to a human in need thereof a therapeutically effective amount of a compound of Formula (I) or a pharmaceutically acceptable salt thereof.

### DETAILED DESCRIPTION OF THE INVENTION

As used herein, “a compound of the invention” includes all solvates, complexes, polymorphs, radiolabelled derivatives, stereoisomers, tautomers and optical isomers of the compounds of formula (I) and salts thereof.

As used herein, the term “effective amount” means that amount of a drug or pharmaceutical agent that will elicit the biological or medical response of a tissue, system, animal or human that is being sought, for instance, by a researcher or clinician. Furthermore, the term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function.

As used herein, the term “pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

The compounds of the invention may exist in solid or liquid form. In solid form, compound of the invention may exist in a continuum of solid states ranging from fully amorphous to fully crystalline. The term ‘amorphous’ refers to a state in which the material lacks long range order at the molecular level and, depending upon the temperature, may exhibit the physical properties of a solid or a liquid. Typically such materials do not give distinctive X-ray diffraction patterns and, while exhibiting the properties of a solid, are more formally described as a liquid. Upon heating, a change from solid to liquid properties occurs which is characterized by a change of state, typically second order (‘glass transition’). The term ‘crystalline’ refers to a solid phase in which the material has a regular ordered internal structure at the molecular level and gives a distinctive X-ray diffraction pattern with defined peaks. Such materials when heated sufficiently will also exhibit the properties of a liquid, but the change from solid to liquid is characterized by a phase change, typically first order (‘melting point’).

The compound of formula (I) may exist in solvated and unsolvated forms. As used herein, the term “solvate” refers to a complex of variable stoichiometry formed by a solute (in this invention, a compound of formula (I) or a salt) and a solvent. Such solvents for the purpose of the invention may not interfere with the biological activity of the solute. The skilled artisan will appreciate that pharmaceutically acceptable solvates may be formed for crystalline compounds wherein solvent molecules are incorporated into the crystalline lattice during crystallization. The incorporated solvent molecules may be water molecules or non-aqueous such as ethanol, isopropanol, DMSO, acetic acid, ethanolamine, and ethyl acetate molecules. Crystalline lattice incorporated with water molecules are typically referred to as “hydrates”. Hydrates include stoichiometric hydrates as well as compositions containing variable amounts of water. The present invention includes all such solvates.

The compounds of the invention may have the ability to crystallize in more than one form, a characteristic, which is known as polymorphism, and it is understood that such polymorphic forms (“polymorphs”) are within the scope of the invention. Polymorphism generally can occur as a response to changes in temperature or pressure or both and can also result from variations in the crystallization process. Polymorphs can be distinguished by various physical characteristics known in the art such as x-ray diffraction patterns, solubility and melting point.

It is also noted that the compounds of formula (I) may form tautomers. It is understood that all tautomers and mixtures of tautomers of the compounds of the present invention are included within the scope of the compounds of the present invention.

Compounds binding to the target kinases Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC) are known in the art.

In one aspect of the present invention the linker is a chemical linker group.

In one aspect the linker group is 4-20 atoms in shortest length.

In one aspect the linker group Is a straight chain alkylene group of 4-20 carbon atoms in which one or more carbon atoms is replaced by a group independently selected from —O—, —NH—, —N(CH₃)—, —CO—, piperidine, piperazine, pyrimidine, pyridine.

In one aspect the linker is (in the direction Kinase binder-cereblon binder):

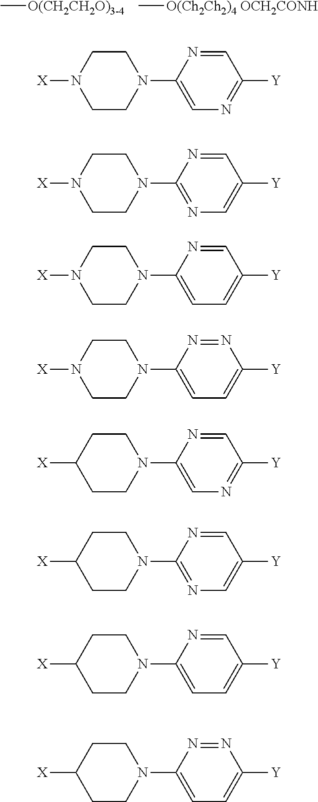

wherein X is —O(CH₂CH₂)₀₋₄—,

### and Y is —CONH—, —O— or —CO—.

In further aspect of the invention the Cereblon binding moiety is a compound thalidomide (7), pomalidomide (8) and lenalidomide (9):

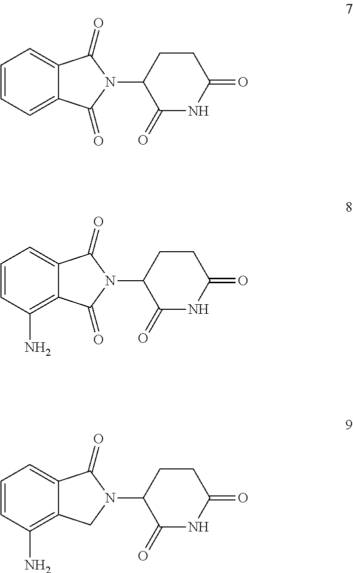 [SMILES: O=C1CCC(N2C(=O)c3ccccc3C2=O)C(=O)N1 ; Nc1cccc2c1C(=O)N(C1CCC(=O)NC1=O)C2=O ; Nc1cccc2c1CN(C1CCC(=O)NC1=O)C2=O]

In a further aspect of the invention there is provided a Protac compound comprising the compound of Formula (I) linked via the linker to a compound which binds to a target protein, where said target protein is Adaptor-associated protein kinase 1 (AAK1).

In a further aspect the target protein is Abelson murine leukemia viral oncogene homolog 1 (ABL1).

In a further aspect the target protein is Aurora Kinase A (AURKA).

In a further aspect the target protein is Aurora Kinase B (AURKB).

In a further aspect the target protein is Bruton's Tyrosine Kinase (BTK).

In a further aspect the target protein is Interleukin-1 receptor-associated kinase 3 (IRAK3).

In a further aspect the target protein is Protein tyrosine kinase 2 beta (PTK2B).

In a further aspect the target protein is Tyrosine-protein kinase Tec (TEC).

In a further aspect the target protein is Cyclin G-associated kinase (GAK).

In a further aspect the target protein is Large Tumour suppressor 1 Kinase (LATS1).

In a further aspect the target protein is Focal Adhesion Kinase (PTK2).

In a further aspect the target protein is Ribosomal protein S6 kinase alpha-1 (RPS6KA1).

In a further aspect the target protein is Mitogen-activated protein kinase 9 (MAPK9).

In a further aspect the target protein is Protein kinase AMP-activated alpha-1 (PRKAA1).

In a further aspect the target protein is Ribosomal protein S6 kinase alpha-3 (RPS6KA3).

The compounds of Formula (I) may be in the form of a salt.

Typically, the salts of the present invention are pharmaceutically acceptable salts. Salts encompassed within the term “pharmaceutically acceptable salts” refer to non-toxic salts of the compounds of this invention. For a review on suitable salts see Berge et al, J. Pharm. Sci. 1977, 66, 1-19.

Suitable pharmaceutically acceptable salts can include acid addition salts. A pharmaceutically acceptable acid addition salt can be formed by reaction of a compound of formula (I) with a suitable inorganic or organic acid (such as hydrobromic, hydrochloric, sulfuric, nitric, phosphoric, p-toluenesulfonic, benzenesulfonic, methanesulfonic, ethanesulfonic, naphthalenesulfonic such as 2-naphthalenesulfonic), optionally in a suitable solvent such as an organic solvent, to give the salt which is usually isolated for example by crystallisation and filtration. A pharmaceutically acceptable acid addition salt of a compound of formula (I) can comprise or be for example a hydrobromide, hydrochloride, sulfate, nitrate, phosphate, p-toluenesulfonate, benzenesulfonate, methanesulfonate, ethanesulfonate, naphthalenesulfonate (e.g. 2-naphthalenesulfonate) salt.

Other non-pharmaceutically acceptable salts, e.g. trifluoroacetates, may be used, for example in the isolation of compounds of the invention, and are included within the scope of this invention.

The invention includes within its scope all possible stoichiometric and non-stoichiometric forms of the compounds of formula (I).

While it is possible that, for use in therapy, the compound of the invention may be administered as the raw chemical, it is possible to present the compound of the invention as the active ingredient as a pharmaceutical composition. Such compositions can be prepared in a manner well known in the pharmaceutical art and comprise at least one active compound. Accordingly, the invention further provides pharmaceutical compositions comprising a compound of the invention and one or more pharmaceutically acceptable excipients. The excipient(s) must be acceptable in the sense of being compatible with the other ingredients of the composition and not deleterious to the recipient thereof. In accordance with another aspect of the invention there is also provided a process for the preparation of a pharmaceutical composition including the agent, or pharmaceutically acceptable salts thereof, with one or more pharmaceutically acceptable excipients. The pharmaceutical composition can be for use in the treatment and/or prophylaxis of any of the conditions described herein.

Generally, the compound of the invention is administered in a pharmaceutically effective amount. The amount of the compound actually administered will typically be determined by a physician, in the light of the relevant circumstances, including the condition to be treated, the chosen route of administration, the actual compound-administered, the age, weight, and response of the individual patient, the severity of the patient's symptoms, and the like.

Pharmaceutical compositions may be presented in unit dose forms containing a predetermined amount of active ingredient per unit dose. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient, vehicle or carrier. Typical unit dosage forms include prefilled, premeasured ampules or syringes of the liquid compositions or pills, tablets, capsules or the like in the case of solid compositions.

Preferred unit dosage compositions are those containing a daily dose or sub-dose, or an appropriate fraction thereof, of an active ingredient. Such unit doses may therefore be administered once or more than once a day. Such pharmaceutical compositions may be prepared by any of the methods well known in the pharmacy art.

Pharmaceutical compositions may be adapted for administration by any appropriate route, for example by the oral (including buccal or sublingual), rectal, inhaled, intranasal, topical (including buccal, sublingual or transdermal), vaginal or parenteral (including subcutaneous, intramuscular, intravenous or intradermal) route. Such compositions may be prepared by any method known in the art of pharmacy, for example by bringing into association the active ingredient with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

For instance, for oral administration in the form of a tablet or capsule, the active drug component can be combined with an oral, non-toxic pharmaceutically acceptable inert excipient such as ethanol, glycerol, water and the like. Powders are prepared by reducing the compound to a suitable fine size and mixing with a similarly prepared pharmaceutical excipient such as an edible carbohydrate, as, for example, starch or mannitol. Flavouring, preservative, dispersing and colouring agent can also be present.

Capsules are made by preparing a powder mixture, as described above, and filling formed gelatin sheaths. Excipients including glidants and lubricants such as colloidal silica, talc, magnesium stearate, calcium stearate or solid polyethylene glycol can be added to the powder mixture before the filling operation. A disintegrating or solubilizing agent such as agar-agar, calcium carbonate or sodium carbonate can also be added to improve the availability of the medicament when the capsule is ingested.

Moreover, when desired or necessary, excipients including suitable binders, glidants, lubricants, sweetening agents, flavours, disintegrating agents and colouring agents can also be incorporated into the mixture. Suitable binders include starch, gelatin, natural sugars such as glucose or beta-lactose, corn sweeteners, natural and synthetic gums such as acacia, tragacanth or sodium alginate, carboxymethylcellulose, polyethylene glycol, waxes and the like. Lubricants used in these dosage forms include sodium oleate, sodium stearate, magnesium stearate, sodium benzoate, sodium acetate, sodium chloride and the like. Disintegrators include, without limitation, starch, methyl cellulose, agar, bentonite, xanthan gum and the like. Tablets are formulated, for example, by preparing a powder mixture, granulating or slugging, adding a lubricant and disintegrant and pressing into tablets. A powder mixture is prepared by mixing the compound, suitably comminuted, with a diluent or base as described above, and optionally, with a binder such as carboxymethylcellulose, an aliginate, gelatin, or polyvinyl pyrrolidone, a solution retardant such as paraffin, a resorption accelerator such as a quaternary salt and/or an absorption agent such as bentonite, kaolin or dicalcium phosphate. The powder mixture can be granulated by wetting with a binder such as syrup, starch paste, acadia mucilage or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material and a polish coating of wax can be provided. Dyestuffs can be added to these coatings to distinguish different unit dosages.

Oral fluids such as solution, suspensions, syrups and elixirs can be prepared in dosage unit form so that a given quantity contains a predetermined amount of the compound. Syrups can be prepared by dissolving the compound in a suitably flavoured aqueous solution, while elixirs are prepared through the use of a non-toxic alcoholic vehicle. Suspensions can be formulated by dispersing the compound in a non-toxic vehicle. Solubilizers and emulsifiers such as ethoxylated isostearyl alcohols and polyoxy ethylene sorbitol ethers, preservatives, flavor additive such as peppermint oil or natural sweeteners or saccharin or other artificial sweeteners, and the like can also be added.

Where appropriate, dosage unit compositions for oral administration can be microencapsulated. The composition can also be prepared to prolong or sustain the release as for example by coating or embedding particulate material in polymers, wax or the like.

The compounds of the invention may also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Pharmaceutical compositions adapted for transdermal administration may be presented as discrete patches intended to remain in intimate contact with the epidermis of the recipient for a prolonged period of time.

Pharmaceutical compositions adapted for topical administration may be formulated as ointments, creams, suspensions, lotions, powders, solutions, pastes, gels, sprays, aerosols or oils.

For treatments of the eye or other external tissues, for example mouth and skin, the compositions are preferably applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical compositions adapted for topical administrations to the eye include eye drops wherein the active ingredient is dissolved or suspended in a suitable carrier, especially an aqueous solvent.

Pharmaceutical compositions adapted for topical administration in the mouth include lozenges, pastilles and mouth washes.

Pharmaceutical compositions adapted for rectal administration may be presented as suppositories, rectal foams, rectal gels or as enemas.

Dosage forms for nasal or inhaled administration may conveniently be formulated as aerosols, solutions, suspensions drops, gels or dry powders.

Pharmaceutical compositions adapted for vaginal administration may be presented as pessaries, tampons, creams, gels, pastes, foams or spray formulations.

Pharmaceutical compositions adapted for parental administration include aqueous and non-aqueous sterile injection solutions which may contain anti-oxidants, buffers, bacteriostats and solutes which render the composition isotonic with the blood of the intended recipient; and aqueous and non-aqueous sterile suspensions which may include suspending agents and thickening agents. The compositions may be presented in unit-dose or multi-dose containers, for example sealed ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets.

It should be understood that in addition to the ingredients particularly mentioned above, the compositions may include other agents conventional in the art having regard to the type of formulation in question, for example those suitable for oral administration may include flavouring agents.

In one aspect the pharmaceutical composition is suitable for oral or rectal administration for non systemic or local delivery to the GI tract, or is formulated for subcutaneous delivery.

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The compounds of formula (I) and pharmaceutically acceptable salts thereof may be employed alone or in combination with other therapeutic agents. The compounds of formula (I) and pharmaceutically acceptable salts thereof and the other pharmaceutically active agent(s) may be administered together or separately and, when administered separately, administration may occur simultaneously or sequentially, in any order. by any convenient route in separate or combined pharmaceutical compositions.

The amounts of the compound(s) of formula (I) or pharmaceutically acceptable salt(s) thereof and the other pharmaceutically active agent(s) and the relative timings of administration will be selected in order to achieve the desired combined therapeutic effect. The compounds of the present invention and further therapeutic agent(s) may be employed in combination by administration simultaneously in a unitary pharmaceutical composition including both compounds. Alternatively, the combination may be administered separately in separate pharmaceutical compositions, each including one of the compounds in a sequential manner wherein, for example, the compound of the invention is administered first and the other second and visa versa. Such sequential administration may be close in time (e.g. simultaneously) or remote in time. Furthermore, it does not matter if the compounds are administered in the same dosage form, e.g. one compound may be administered topically and the other compound may be administered orally. Suitably, both compounds are administered orally.

The combinations may be presented as a combination kit. By the term “combination kit” “or kit of parts” as used herein is meant the pharmaceutical composition or compositions that are used to administer the combination according to the invention. When both compounds are administered simultaneously, the combination kit can contain both compounds in a single pharmaceutical composition, such as a tablet, or in separate pharmaceutical compositions. When the compounds are not administered simultaneously, the combination kit will contain each compound in separate pharmaceutical compositions either in a single package or in separate pharmaceutical compositions in separate packages.

The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

When the combination is administered separately in a sequential manner wherein one is administered first and the other second or vice versa, such sequential administration may be close in time or remote in time. For example, administration of the other agent several minutes to several dozen minutes after the administration of the first agent, and administration of the other agent several hours to several days after the administration of the first agent are included, wherein the lapse of time is not limited, For example, one agent may be administered once a day, and the other agent may be administered 2 or 3 times a day, or one agent may be administered once a week, and the other agent may be administered once a day and the like.

It will be clear to a person skilled in the art that, where appropriate, the other therapeutic ingredients(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art.

When the compound of formula (I) is used in combination with a second therapeutic agent active against the same disease, condition or disorder, the dose of each compound may differ from that when the compound is used alone. Appropriate doses will be readily appreciated by those skilled in the art.

In one embodiment the mammal in the methods and uses of the present invention is a human.

We have found that the Cereblon containing Protac compounds of the present invention, or a pharmaceutically acceptable salt thereof, or pharmaceutical compositions containing them, are capable of degrading the target protein.

Accordingly, the compounds of the present invention are expected to be potentially useful agents in the treatment of diseases or medical conditions mediated alone or in part by the target protein. Provided herein are methods of treatment or prevention of diseases, disorders and conditions mediated by the target protein. A method may comprise administering to a subject, e.g. a subject in need thereof, a therapeutically effective amount of a compound of the invention.

Thus in one aspect there is provided a compound of the invention for use in therapy.

Thus in one aspect there is provided a compound of the invention for use in treating disorders mediated by the target protein.

Thus in one aspect there is provided the use of a compound of the invention in the manufacture of a medicament for treating disorders mediated by the target protein.

In a further aspect there is provided a method of treatment of, disorders mediated by the target protein in a mammal comprising administering a therapeutically effective amount of a compound of the invention.

Disorders mediated by the target protein as used herein, denotes a condition or disorder which can be treated by modulating the function or activity of a target protein in a subject, wherein treatment comprises prevention, partial alleviation or cure of the condition or disorder. Modulation may occur locally, for example, within certain tissues of the subject, or more extensively throughout a subject being treated for such a condition or disorder.

A therapeutically effective amount of the agent will depend upon a number of factors including, for example, the age and weight of the subject, the precise condition requiring treatment and its severity, the nature of the formulation, and the route of administration, and will ultimately be at the discretion of the attendant physician or veterinarian. In particular, the subject to be treated is a mammal, particularly a human.

The agent may be administered in a daily dose. This amount may be given in a single dose per day or more usually in a number (such as two, three, four, five or six) of sub-doses per day such that the total daily dose is the same.

Suitably, the amount of the compound of the invention administered according to the present invention will be an amount selected from 0.01 mg to 1 g per day (calculated as the free or unsalted compound).

The compounds of formula (I) and pharmaceutically acceptable salts thereof may be employed alone or in combination with other therapeutic agents. The compounds of formula (I) and pharmaceutically acceptable salts thereof and the other pharmaceutically active agent(s) may be administered together or separately and, when administered separately, administration may occur simultaneously or sequentially, in any order. by any convenient route in separate or combined pharmaceutical compositions.

The amounts of the compound(s) of formula (I) or pharmaceutically acceptable salt(s) thereof and the other pharmaceutically active agent(s) and the relative timings of administration will be selected in order to achieve the desired combined therapeutic effect. The compounds of the present invention and further therapeutic agent(s) may be employed in combination by administration simultaneously in a unitary pharmaceutical composition including both compounds. Alternatively, the combination may be administered separately in separate pharmaceutical compositions, each including one of the compounds in a sequential manner wherein, for example, the compound of the invention is administered first and the other second and visa versa. Such sequential administration may be close in time (e.g. simultaneously) or remote in time. Furthermore, it does not matter if the compounds are administered in the same dosage form, e.g. one compound may be administered topically and the other compound may be administered orally. Suitably, both compounds are administered orally.

The combinations may be presented as a combination kit. By the term “combination kit” “or kit of parts” as used herein is meant the pharmaceutical composition or compositions that are used to administer the combination according to the invention. When both compounds are administered simultaneously, the combination kit can contain both compounds in a single pharmaceutical composition, such as a tablet, or in separate pharmaceutical compositions. When the compounds are not administered simultaneously, the combination kit will contain each compound in separate pharmaceutical compositions either in a single package or in separate pharmaceutical compositions in separate packages.

The combination kit can also be provided by instruction, such as dosage and administration instructions. Such dosage and administration instructions can be of the kind that are provided to a doctor, for example by a drug product label, or they can be of the kind that are provided by a doctor, such as instructions to a patient.

When the combination is administered separately in a sequential manner wherein one is administered first and the other second or vice versa, such sequential administration may be close in time or remote in time. For example, administration of the other agent several minutes to several dozen minutes after the administration of the first agent, and administration of the other agent several hours to several days after the administration of the first agent are included, wherein the lapse of time is not limited, For example, one agent may be administered once a day, and the other agent may be administered 2 or 3 times a day, or one agent may be administered once a week, and the other agent may be administered once a day and the like.

It will be clear to a person skilled in the art that, where appropriate, the other therapeutic ingredients(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

When combined in the same composition it will be appreciated that the two compounds must be stable and compatible with each other and the other components of the composition and may be formulated for administration. When formulated separately they may be provided in any convenient composition, conveniently, in such a manner as known for such compounds in the art. When the compound of formula (I) is used in combination with a second therapeutic agent active against the same disease, condition or disorder, the dose of each compound may differ from that when the compound is used alone. Appropriate doses will be readily appreciated by those skilled in the art.

In one embodiment the mammal in the methods and uses of the present invention is a human.

The compounds of the invention may be particularly useful for treatment kinase-mediated disorders, particularly inflammatory disorders, many cancers and other proliferative diseases.

In one aspect the disorder is inflammation.

Inflammation represents a group of vascular, cellular and neurological responses to trauma. Inflammation can be characterised as the movement of inflammatory cells such as monocytes, neutrophils and granulocytes into the tissues. This is usually associated with reduced endothelial barrier function and oedema into the tissues. Inflammation can be classified as either acute or chronic. Acute inflammation is the initial response of the body to harmful stimuli and is achieved by the increased movement of plasma and leukocytes from the blood into the injured tissues. A cascade of biochemical event propagates and matures the inflammatory response, involving the local vascular system, the immune system, and various cells within the injured tissue. Prolonged inflammation, known as chronic inflammation, leads to a progressive shift in the type of cells which are present at the site of inflammation and is characterised by simultaneous destruction and healing of the tissue from the inflammatory process.

When occurring as part of an immune response to infection or as an acute response to trauma, inflammation can be beneficial and is normally self-limiting. However, inflammation can be detrimental under various conditions. This includes the production of excessive inflammation in response to infectious agents, which can lead to significant organ damage and death (for example, in the setting of sepsis). Moreover, chronic inflammation is generally deleterious and is at the root of numerous chronic diseases, causing severe and irreversible damage to tissues. In such settings, the immune response is often directed against self-tissues (autoimmunity), although chronic responses to foreign entities can also lead to bystander damage to self tissues.

The aim of anti-inflammatory therapy is therefore to reduce this inflammation, to inhibit autoimmunity when present and to allow for the physiological process or healing and tissue repair to progress.

The compound of formula (I) may be used to treat inflammation of any tissue and organs of the body, including musculoskeletal inflammation, vascular inflammation, neural inflammation, digestive system inflammation, ocular inflammation, inflammation of the reproductive system, and other inflammation, as exemplified below.

Musculoskeletal inflammation refers to any inflammatory condition of the musculoskeletal system, particularly those conditions affecting skeletal joints, including joints of the hand, wrist, elbow, shoulder, jaw, spine, neck, hip, knew, ankle, and foot, and conditions affecting tissues connecting muscles to bones such as tendons. Examples of musculoskeletal inflammation which may be treated with compounds of formula (I) include arthritis (including, for example, osteoarthritis, rheumatoid arthritis, psoriatic arthritis, ankylosing spondylitis, acute and chronic infectious arthritis, arthritis associated with gout and pseudogout, and juvenile idiopathic arthritis), tendonitis, synovitis, tenosynovitis, bursitis, fibrositis (fibromyalgia), epicondylitis, myositis, and osteitis (including, for example, Paget's disease, osteitis pubis, and osteitis fibrosa cystic).

Ocular inflammation refers to inflammation of any structure of the eye, including the eye lids.

Examples of ocular inflammation which may be treated with the compounds of formula (I) include blepharitis, blepharochalasis, conjunctivitis, dacryoadenitis, keratitis, keratoconjunctivitis sicca (dry eye), scleritis, trichiasis, and uveitis.

Examples of inflammation of the nervous system which may be treated with the compounds of formula (I) include encephalitis, Guillain-Barre syndrome, meningitis, neuromyotonia, narcolepsy, multiple sclerosis, myelitis and schizophrenia.

Examples of inflammation of the vasculature or lymphatic system which may be treated with the compounds of formula (I) include arthrosclerosis, arthritis, phlebitis, vasculitis, and lymphangitis.

Examples of inflammatory conditions of the digestive system which may be treated with the compounds of formula (I) include cholangitis, cholecystitis, enteritis, enterocolitis, gastritis, gastroenteritis, inflammatory bowel disease (such as Crohn's disease and ulcerative colitis), ileitis, and proctitis.

Examples of inflammatory conditions of the reproductive system which may be treated with the compounds of formula (I) include cervicitis, chorioamnionitis, endometritis, epididymitis, omphalitis, oophoritis, orchitis, salpingitis, tubo-ovarian abscess, urethritis, vaginitis, vulvitis, and vulvodynia.

The compound of formula (I) may be used to treat autoimmune conditions having an inflammatory component. Such conditions include acute disseminated alopecia universalise, Behcet's disease, Chagas' disease, chronic fatigue syndrome, dysautonomia, encephalomyelitis, ankylosing spondylitis, aplastic anemia, hidradenitis suppurativa, autoimmune hepatitis, autoimmune oophoritis, celiac disease, Crohn's disease, diabetes mellitus type 1, giant cell arteritis, goodpasture's syndrome, Grave's disease, Guillain-Barre syndrome, Hashimoto's disease, Henoch-Schönlein purpura, Kawasaki's disease, lupus erythematosus, microscopic colitis, microscopic polyarteritis, mixed connective tissue disease, multiple sclerosis, myasthenia gravis, opsocionus myoclonus syndrome, optic neuritis, ord's thyroiditis, pemphigus, polyarteritis nodosa, polymyalgia, rheumatoid arthritis, Reiter's syndrome, Sjogren's syndrome, temporal arteritis, Wegener's granulomatosis, warm autoimmune haemolytic anemia, interstitial cystitis, lyme disease, morphea, psoriasis, sarcoidosis, scleroderma, ulcerative colitis, and vitiligo.

The compound of formula (I) may be used to treat T-cell mediated hypersensitivity diseases having an inflammatory component. Such conditions include contact hypersensitivity, contact dermatitis (including that due to poison ivy), uticaria, skin allergies, respiratory allergies (hayfever, allergic rhinitis) and gluten-sensitive enteropathy (Celliac disease).

Other inflammatory conditions which may be treated with the agents include, for example, appendicitis, dermatitis, dermatomyositis, endocarditis, fibrositis, gingivitis, glossitis, hepatitis, hidradenitis suppurativa, iritis, laryngitis, mastitis, myocarditis, nephritis, otitis, pancreatitis, parotitis, percarditis, peritonoitis, pharyngitis, pleuritis, pneumonitis, prostatistis, pyelonephritis, and stomatisi, transplant rejection (involving organs such as kidney, liver, heart, lung, pancreas (e.g., islet cells), bone marrow, cornea, small bowel, skin allografts, skin homografts, and heart valve xengrafts, sewrum sickness, and graft vs host disease), acute pancreatitis, chronic pancreatitis, acute respiratory distress syndrome, Sexary's syndrome, congenital adrenal hyperplasis, nonsuppurative thyroiditis, hypercalcemia associated with cancer, pemphigus, bullous dermatitis herpetiformis, severe erythema multiforme, exfoliative dermatitis, seborrheic dermatitis, seasonal or perennial allergic rhinitis, bronchial asthma, contact dermatitis, astopic dermatitis, drug hypersensistivity reactions, allergic conjunctivitis, keratitis, herpes zoster ophthalmicus, iritis and oiridocyclitis, chorioretinitis, optic neuritis, symptomatic sarcoidosis, fulminating or disseminated pulmonary tuberculosis chemotherapy, idiopathic thrombocytopenic purpura in adults, secondary thrombocytopenia in adults, acquired (autroimmine) haemolytic anemia, leukaemia and lymphomas in adults, acute leukaemia of childhood, regional enteritis, autoimmune vasculitis, multiple sclerosis, chronic obstructive pulmonary disease, solid organ transplant rejection, sepsis. Preferred treatments include treatment of transplant rejection, rheumatoid arthritis, psoriatic arthritis, multiple sclerosis, Type 1 diabetes, asthma, inflammatory bowel disease, systemic lupus erythematosis, psoriasis, chronic obstructive pulmonary disease, and inflammation accompanying infectious conditions (e.g., sepsis).

Treatment of kinase-mediated diseases or disorders, or more broadly, treatment of immune mediated diseases including, but not limited to, allergic diseases, autoimmune diseases, prevention of transplant rejection and the like, may be achieved using a compound of this invention as a monotherapy, or in dual or multiple combination therapy, with or include one or more other therapeutic agents, for example selected from NSAIDS, corticosteroids, COX-2 inhibitors, cytokine inhibitors, anti-TNF agents, inhibitors oncostatin M, anti-malarials, immunsuppressive and cytostatics.

In one aspect the disorder is cancer.

Examples of cancer diseases and conditions in which compounds of formula (I), or pharmaceutically acceptable salts or solvates thereof may have potentially beneficial antitumour effects include, but are not limited to, cancers of the lung, bone, pancreas, skin, head, neck, uterus, ovaries, stomach, colon, breast, esophagus, small intestine, bowel, endocrine system, thyroid glad, parathyroid gland, adrenal gland, urethra, prostate, penis, testes, ureter, bladder, kidney or liver; rectal cancer; cancer of the anal region; carcinomas of the fallopian tubes, endometrium, cervix, vagina, vulva, renal pelvis, renal cell; sarcoma of soft tissue; myxoma; rhabdomyoma; fibroma; lipoma; teratoma; cholangiocarcinoma; hepatoblastoma; angiosarcoma; hemagioma; hepatoma; fibrosarcoma; chondrosarcoma; myeloma; chronic or acute leukemia; lymphocytic lymphomas; primary CNS lymphoma; neoplasms of the CNS; spinal axis tumours; squamous cell carcinomas; synovial sarcoma; malignant pleural mesotheliomas; brain stem glioma; pituitary adenoma; bronchial adenoma; chondromatous hanlartoma; inesothelioma; Hodgkin's Disease or a combination of one or more of the foregoing cancers. In one aspect the cancer is breast cancer.

The compounds of the present invention may also be useful in the treatment of one or more diseases afflicting mammals which are characterized by cellular proliferation in the area of disorders associated with neo-vascularization and/or vascular permeability including blood vessel proliferative disorders including arthritis (rheumatoid arthritis) and restenosis; fibrotic disorders including hepatic cirrhosis and atherosclerosis; mesangial cell proliferative disorders include glomerulonephritis, diabetic nephropathy, malignant nephrosclerosis, thrombotic microangiopathy syndromes, proliferative retinopathies, organ transplant rejection and glomerulopathies; and metabolic disorders include psoriasis, diabetes mellitus, chronic wound healing, inflammation and neurodegenerative diseases.

In one embodiment, the compound of compound of formula (I) or a pharmaceutically acceptable salt thereof may be employed with other therapeutic methods of cancer treatment. In particular, in anti-neoplastic therapy, combination therapy with other chemotherapeutic, hormonal, antibody agents as well as surgical and/or radiation treatments other than those mentioned above are envisaged.

In one embodiment, the further anti-cancer therapy is surgical and/or radiotherapy.

In one embodiment, the further anti-cancer therapy is at least one additional anti-neoplastic agent.

Any anti-neoplastic agent that has activity versus a susceptible tumor being treated may be utilized in the combination. Typical anti-neoplastic agents useful include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracyclins, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine angiogenesis inhibitors; immunotherapeutic agents; proapoptotic agents; and cell cycle signaling inhibitors.

In a further aspect there is provided a pharmaceutical composition comprising a combination comprising a compound of formula (I) or a pharmaceutically acceptable salt thereof and at least one further therapeutic agent useful in the treatment of a disease mediated by inhibition of the target protein and one or more of pharmaceutically acceptable excipients.

### General Synthetic Methods

Compounds of general formula (I) may be prepared by methods known in the art of organic synthesis. In all of the methods, it is well understood that protecting groups for sensitive or reactive groups may be employed where necessary in accordance with general principles of chemistry. Protecting groups are manipulated according to standard methods of organic synthesis (T. W. Green and P. G. M. Wuts (1999) Protective Groups in Organic Synthesis, 3^{rd} edition, John Wiley & Sons). These groups are removed at a convenient stage of the compound synthesis using methods that are readily apparent to those skilled in the art. The selection of processes as well as the reaction conditions and order of their execution shall be consistent with the preparation of compounds of Formula (I).

In particular, methods for preparing CEREBLON compounds included in the present invention can be found in WO2016024286 or are available commercially.

### Promiscuous CEREBLON Protac Synthesis

A promiscuous kinase binder was prepared as described in WO2013/75167A1 (or RSC Adv., 2015, 5, 93433-93437)

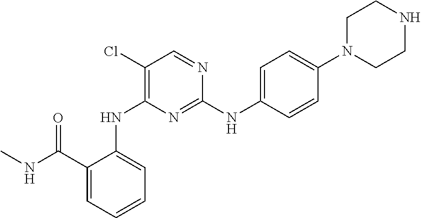 [SMILES: CNC(=O)c1ccccc1Nc1nc(Nc2ccc(N3CCNCC3)cc2)ncc1Cl]

### 14-(4-(4-((5-Chloro-4-((2-(methylcarbamoyl)phenyl)amino)pyrimidin-2-yl)amino)phenyl) piperazin-1-yl)-3,6,9,12-tetraoxatetradecan-1-oic acid

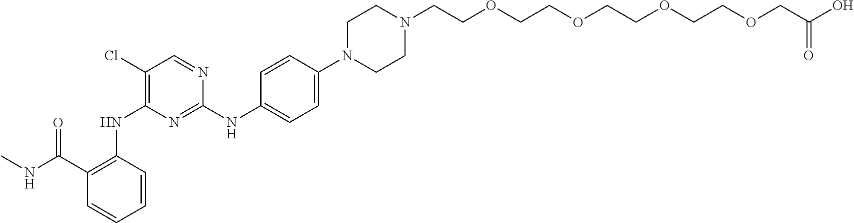 [SMILES: CNC(=O)c1ccccc1Nc1nc(Nc2ccc(N3CCN(CCOCCOCCOCCOCC(=O)O)CC3)cc2)ncc1Cl]

A solution of 2-((5-chloro-2-((4-(piperazin-1-yl)phenyl)amino)pyrimidin-4-yl)amino)-N-methylbenzamide (150 mg, 0.343 mmol), methyl 14-chloro-3,6,9,12-tetraoxatetradecanoate (117 mg, 0.411 mmol), sodium iodide (52 mg, 0.347 mmol) and diisopropylamine (0.179 mL, 1.03 mmol) in DMF (2.5 mL) was heated at 100° C. for 24 h. The mixture was diluted with n-BuOH (15 mL) and water (30 mL), then the phases were separated. The aqueous solution was back-extracted with n-BuOH (15 mL), then the organic layers were combined and evaporated in vacuo. The residue was dissolved in MeOH (5 mL), then a solution of LiOH (82 mg, 3.43 mmol) in water (1 mL) was added and the mixture stirred at room temperature for 2 h. The reaction mixture was evaporated in vacuo, then dissolved in minimal DMSO and purified by reverse phase (C18) chromatography using a 0-50% acetonitrile-water (+0.1% ammonium bicarbonate modifier) gradient over 12 column volumes. The appropriate fractions were combined and evaporated in vacuo to give the required product (153 mg, 67% yield) as a gold solid.

LCMS (High pH modifier) (ES+ve) m/z 672.2 (M+H)⁺ Rt 0.78 min (>95% pure)

### 14-(4-(4-((5-Chloro-4-((2-(methylcarbamoyl)phenyl)amino)pyrimidin-2-yl)amino)phenyl)piperazin-1-yl)-N-(2-(2,6-dioxopiperidin-3-yl)-1-oxoisoindolin-4-yl)-3,6,9,12-tetraoxatetradecan-1-amide formate

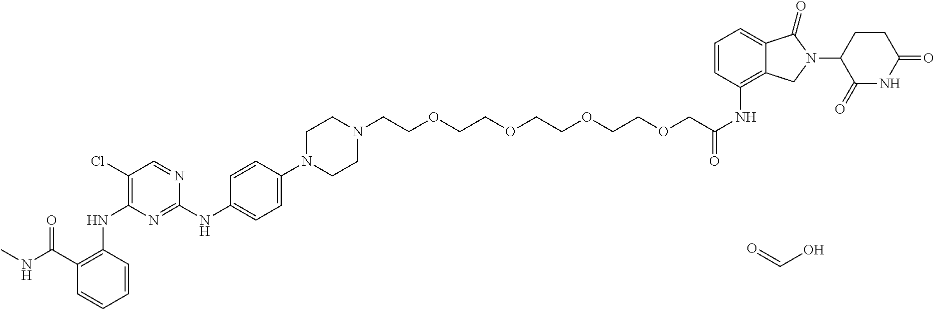 [SMILES: CNC(=O)c1ccccc1Nc1nc(Nc2ccc(N3CCN(CCOCCOCCOCCOCC(=O)Nc4cccc5c4CN(C4CCC(=O)NC4=O)C5=O)CC3)cc2)ncc1Cl ; O=CO]

To a stirred solution of 14-(4-(4-((5-chloro-4-((2-(methylcarbamoyl)phenyl)amino)pyrimidin-2-yl)amino)phenyl)piperazin-1-yl)-3,6,9,12-tetraoxatetradecanoic acid (109 mg, 0.162 mmol), 3-(4-amino-1-oxoisoindolin-2-yl)piperidine-2,6-dione (46.2 mg, 0.178 mmol) and diisopropylamine (0.085 mL, 0.486 mmol) in DMF (1.5 mL) was added HATU (74.0 mg, 0.195 mmol) and the mixture stirred at rt for 1 h. The reaction mixture was directly purified by mass directed auto purification (formic acid modifier gradient), then the appropriate fractions concentrated under a stream of nitrogen to give the required product (57 mg, 37% yield) as a yellow solid.

LCMS (Formic acid modifier) (ES+ve) m/z 913.3 (M−formate)⁺ Rt 0.60 min (>95% pure)

LCMS (High pH modifier) (ES+ve) m/z 1116.4 (M+H)⁺ Rt 1.35 min (>95%

### Cell Treatment for Expression Proteomics Experiment

THP-1 cells were seeded at a concentration of 3×10⁶ cells in T175 flasks with 60 mL growth medium (RPMI1640+10% FBS). 6 μL of a 10× compound solution prepared in growth medium (DMSO), CEREBLON_PROTAC) was added and the cells were treated for the indicated time points (6 or 24 h) at 37° C., 5% CO₂. For harvesting the cells were collected into falcon tubes on ice, centrifuged and washed twice in cold PBS (Life technologies). After the last washing step the supernatant was removed and the pellets were snap-frozen in liquid N₂ and stored at −80° C. and lysed in 2% SDS for 3 min at 95° C. in a thermomixer (Thermo Fisher Scientific), followed by digestion of DNA with Benzonase at 37° C. for 1.5 h. Lysate was cleared by centrifugation an protein concentration in supernatant was determined by BCA assay. Proteins were reduced by DTT and alkylated with iodacetamid and separated on 4-12% NuPAGE (Invitrogen), and stained with colloidal Coomassie (Becher, I. et al. Chemoproteomics Reveals Time-Dependent Binding of Histone Deacetylase Inhibitors to Endogenous Repressor Complexes. ACS Chem. Biol. 9, 1736-1746 (2014) before proceeding to trypsin digestion and mass spectrometric analysis (see below).

### Kinobeads Assays

Competition binding assays were performed by using a modified bead matrix. (Bantscheff, M. et al. Quantitative chemical proteomics reveals mechanisms of action of clinical ABL kinase inhibitors. Nat Biotech 25, 1035-1044 (2007), Werner, T. et al. High-Resolution Enabled TMT 8-plexing. Anal. Chem. 84, 7188-7194 (2012), Bergamini, G. et al. A selective inhibitor reveals PI3Kγ dependence of TH17 cell differentiation. Nat Chem Biol 8, 576-582 (2012). Briefly, 1 ml (5 mg protein) cell extract was pre-incubated with test compound or vehicle for 45 min at 4° C. followed by incubation with kinobeads (35 μl beads per sample) for 1 hour at 4° C. The nonbound fraction was removed by washing the beads with DP buffer (50 mM Tris-HCl, 0.8% (v/v) Igepal-CA630, 5% (v/v) glycerol, 150 mM NaCl, 1.5 mM MgCl₂, 25 mM NaF, 1 mM sodium vanadate, 1 mM dithiothreitol, complete EDTA-free protease inhibitor tablet (Roche), pH 7.5). Proteins retained were eluted with 50 μl 2×SDS sample buffer. Proteins were alkylated with 200 mg/ml iodoacetamide for 30 min, partially separated on 4-12% NuPAGE (Invitrogen), and stained with colloidal Coomassie. CEREBLON_PROTAC were tested at 20, 5, 0.31, 0.078, 0.020, 0.005 μM and the promiscuous kinase-binder was tested at 10, 2.5, 0.63, 0.16, 0.04, 0.01, 0.0024 μM

### Sample Preparation for MS

Gel lanes were cut into three slices covering the entire separation range (˜2 cm) and subjected to in-gel digestion (Bantscheff, M. et al. Quantitative chemical proteomics reveals mechanisms of action of clinical ABL kinase inhibitors. Nat Biotech 25, 1035-1044 (2007). Peptide samples were labeled with 10-plex TMT (TMT10, Thermo Fisher Scientific, Waltham, Mass.) reagents, enabling relative quantification of a broad range of 10 conditions in a single experiment. The labeling reaction was performed in 40 mM triethylammoniumbicarbonate, pH 8.53 at 22° C. and quenched with hydroxylamine. Labeled peptide extracts were combined to a single sample per experiment, and subjected to additional fractionation on an Ultimate3000 (Dionex, Sunnyvale, Calif.) by using reversed-phase chromatography at pH 12 [1 mm Xbridge column (Waters, Milford, Mass.)], as described in Kruse, U. et al. Chemoproteomics-based kinome profiling and target deconvolution of clinical multi-kinase inhibitors in primary chronic lymphocytic leukemia cells. Leukemia 25, 89-100 (2011).

### LC-MS/MS Analysis

Samples were dried in vacuo and resuspended in 0.05% trifluoroacetic acid in water. Of the sample, 50% was injected into an Ultimate3000 nanoRLSC (Dionex, Sunnyvale, Calif.) coupled to a Q Exactive HF (Thermo Fisher Scientific). Peptides were trapped on a 5 mm×300 μm C18 column (Pepmap100, 5 μm, 300 Å, Thermo Fisher Scientific) in water with 0.05% TFA at 60° C. Separation was performed on custom 50 cm×100 μM (ID) reversed-phase columns (Reprosil) at 55° C. Gradient elution was performed from 2% acetonitrile to 40% acetonitrile in 0.1% formic acid and 3.5% DMSO over 2 hours. Samples were online injected into Q-Exactive HF mass spectrometers operating with a data-dependent top 10 method. MS spectra were acquired by using 60.000 resolution and an ion target of 3×10⁶. Higher energy collisional dissociation (HCD) scans were performed with 35% NCE at 30.000 resolution (at m/z 200), and the ion target settings was set to 2×10⁵ so as to avoid coalescence (Werner, T. et al. Ion Coalescence of Neutron Encoded TMT 10-Plex Reporter Ions. Anal. Chem. 86, 3594-3601 (2014).

The instruments were operated with Tune 2.5 and Xcalibur 3.0.63.

### Peptide and Protein Identification

Mascot 2.5.1 (Matrix Science, Boston, Mass.) was used for protein identification using a software lock mass based on the method described by Cox et. al Cox, J., Michalski, A. & Mann, M. Software Lock Mass by Two-Dimensional Minimization of Peptide Mass Errors. Journal of The American Society for Mass Spectrometry 22, 1373-1380 (2011).

The first search was performed with 30 parts per million mass tolerance for peptide precursors and 30 mD (HCD) mass tolerance for fragment ions followed by a final search using recalibrated data with a 10 parts per million mass tolerance for peptide precursors and 20 mD (HCD) mass tolerance for fragment ions. Carbamidomethylation of cysteine residues and TMT modification of lysine residues were set as fixed modifications and methionine oxidation, and N-terminal acetylation of proteins and TMT modification of peptide N-termini were set as variable modifications. The search database consisted of a customized version of the International Protein Index protein sequence database combined with a decoy version of this database created by using a script supplied by Matrix Science. Unless stated otherwise, we accepted protein identifications as follows: (i) For single-spectrum to sequence assignments, we required this assignment to be the best match and a minimum Mascot score of 31 and a 10× difference of this assignment over the next best assignment. Based on these criteria, the decoy search results indicated <1% false discovery rate (FDR). (ii) For multiple spectrum to sequence assignments and using the same parameters, the decoy search results indicate <0.1% FDR.

### Peptide and Protein Quantification

Reporter ion intensities were read from raw data and multiplied with ion accumulation times (the unit is milliseconds) so as to yield a measure proportional to the number of ions; Bantscheff, M. et al. Chemoproteomics profiling of HDAC inhibitors reveals selective targeting of HDAC complexes. Nat Biotech 29, 255-265 (2011). this measure is referred to as ion area Savitski, M. M. et al. Delayed Fragmentation and Optimized Isolation Width Settings for Improvement of Protein Identification and Accuracy of Isobaric Mass Tag Quantification on Orbitrap-Type Mass Spectrometers. Analytical Chemistry 83, 8959-8967 (2011). Spectra matching to peptides were filtered according to the following criteria: mascot ion score >15, signal-to-background of the precursor ion >4, and signal-to-interference >0.5. Savitski, M. M. et al. Targeted data acquisition for improved reproducibility and robustness of proteomic mass spectrometry assays. Journal of the American Society for Mass Spectrometry 21, 1668-1679 (2010).

Fold-changes were corrected for isotope purity as described and adjusted for interference caused by co-eluting nearly isobaric peaks as estimated by the signal-to-interference measure. Savitski, M. M. et al. Measuring and Managing Ratio Compression for Accurate iTRAQ/TMT Quantification. Journal of Proteome Research 12, 3586-3598 (2013). Protein quantification was derived from individual spectra matching to distinct peptides by using a sum-based bootstrap algorithm; 95% confidence intervals were calculated for all protein fold-changes that were quantified with more than three spectra Savitski, M. M. et al. Delayed Fragmentation and Optimized Isolation Width Settings for Improvement of Protein Identification and Accuracy of Isobaric Mass Tag Quantification on Orbitrap-Type Mass Spectrometers. Analytical Chemistry 83, 8959-8967 (2011).

Protein fold changes were only reported for proteins with at least 2 quantified unique peptide matches. Dose-response curves were fitted using R (http://www.r-project.org/) and the drc package (http://www.bioassay.dk), as described previously. Bantscheff, M. et al. Quantitative chemical proteomics reveals mechanisms of action of clinical ABL kinase inhibitors. Nat Biotech 25, 1035-1044 (2007).

All measured half-maximum inhibitory concentration (IC₅₀) values were corrected for the influence of the immobilized ligand on the binding equilibrium using the Cheng-Prusoff relationship. Sharma, K. et al. Proteomics strategy for quantitative protein interaction profiling in cell extracts. Nat Meth 6, 741-744 (2009). Sharma, K. et al. Proteomics strategy for quantitative protein interaction profiling in cell extracts. Nat Meth 6, 741-744 (2009).

### Statistical Analysis

Quantified proteins were divided into bins. The bins are constructed according to the number of quantified spectrum sequence matches. Each bin consists of at least 300 proteins. Once each bin has been completed, the remaining number of proteins is counted; if this number is below 300, the remaining proteins are added to the last completed bin. This data quality-dependent binning strategy is analogous to the procedure described in Cox et al. Savitski, M. M. et al. Delayed Fragmentation and Optimized Isolation Width Settings for Improvement of Protein Identification and Accuracy of Isobaric Mass Tag Quantification on Orbitrap-Type Mass Spectrometers. Analytical Chemistry 83, 8959-8967 (2011). The statistical significance of differences in protein fold change was calculated using a z-test with a robust estimation of the standard deviation (using the 15.87, 50, and 84.13 percentiles) and calculating the P values for all measurements for a specific bin exactly as previously described Cox, J. & Mann, M. MaxQuant enables high peptide identification rates, individualized p.p.b.-range mass accuracies and proteome-wide protein quantification. Nat Biotech 26, 1367-1372 (2008). Subsequently, an adjustment for multiple hypothesis testing was performed for each comparison by using Benjamini-Hochberg (BH) correction. Benjamini, Y. & Hochberg, Y. Controlling the False Discovery Rate: A Practical and Powerful Approach to Multiple Testing. Journal of the Royal Statistical Society. Series B (Methodological) 57, 289-300 (1995). Finally proteins were counted as regulated when having a p-value of 0.05 and changed in their expression in at least 1 replicate by 50%.

| | |
|---|---|
| 24 h 0.1 μM | 24 h 1 μM |
| log2 rel. fc. to vehicle | log2 rel. fc. to vehicle |
| control | control |

| | n = 1 | n = 2 | n = 1 | n = 2 |
|---|---|---|---|---|
| AAK1 | −2.34 | −2.12 | −2.42 | −2.56 |
| PTK2 | −2.29 | −2.92 | −2.42 | −2.20 |
| AURKA | −1.45 | −1.00 | −2.10 | −1.93 |
| PTK2B | −2.34 | −2.50 | −2.09 | −2.18 |
| BTK | −1.65 | −1.67 | −2.02 | −1.86 |
| RPS6KA1 | −1.65 | −1.55 | −1.86 | −1.93 |
| MAPK9 | −0.69 | −0.98 | −1.83 | −1.75 |
| TEC | −1.83 | −2.38 | −1.74 | −1.94 |
| IRAK3 | −2.25 | −1.82 | −1.65 | −1.86 |
| LATS1 | −1.02 | −1.01 | −1.46 | −1.55 |
| ABL1 | −0.72 | −0.70 | −1.43 | −1.63 |
| RPS6KA3 | −0.74 | −0.72 | −1.12 | −1.11 |
| PRKAA1 | −0.38 | −0.74 | −1.06 | −1.35 |
| GAK | −1.13 | −1.35 | −0.93 | −1.13 |
| AURKB | −1.75 | −1.44 | −0.83 | −1.33 |

1. A compound of Formula (I);
Target Protein Binder-Linker-cereblon binder (I)
or a pharmaceutically acceptable salt thereof wherein the target protein is Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC).

2. A compound or pharmaceutically acceptable salt according to claim 1 wherein the linker is a chemical linker group.

3. A compound or pharmaceutically acceptable salt according to claim 1 wherein the linker group is 4-20 atoms in shortest length.

4. A compound or pharmaceutically acceptable salt according to claim 1 wherein linker group Is a straight chain alkylene group of 4-20 carbon atoms in which one or more carbon atoms is replaced by a group independently selected from —O—, —NH—, —N(CH₃)—, —CO—, piperidine, piperazine, pyrimidine, pyridine.

5. A compound or pharmaceutically acceptable salt according to claim 1 wherein the linker is one aspect the linker is (in the direction Kinase binder-cereblon binder): 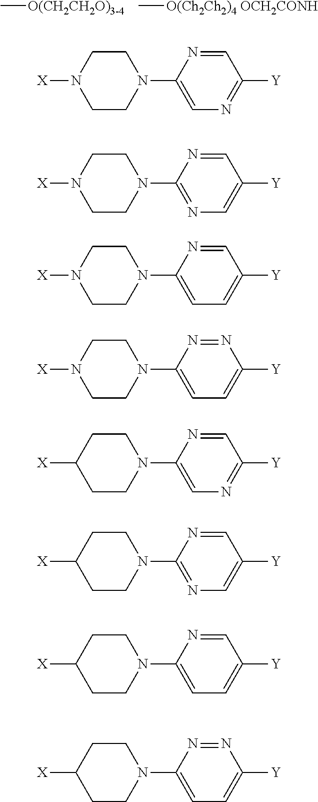
wherein X is —O(CH₂CH₂)₀₋₄—,
and Y is —CONH—, —O— or —CO—.

6. A compound or pharmaceutically acceptable salt according to claim 1 wherein the Cereblon binding moiety is a compound thalidomide (7), pomalidomide (8) or lenalidomide (9):

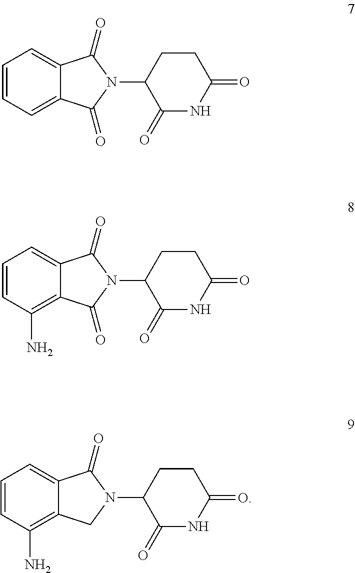 [SMILES: O=C1CCC(N2C(=O)c3ccccc3C2=O)C(=O)N1 ; Nc1cccc2c1C(=O)N(C1CCC(=O)NC1=O)C2=O ; Nc1cccc2c1CN(C1CCC(=O)NC1=O)C2=O]

7. (canceled)

8. (canceled)

9. A pharmaceutical composition comprising a compound of formula (I) or a pharmaceutically acceptable salt thereof according to claim 1 and one or more of pharmaceutically acceptable carriers, diluents and excipients.

10. A method of treating disorders mediated by the target protein in a subject comprising administering a therapeutically effective amount of a compound of formula (I) or a pharmaceutically acceptable salt thereof according to claim 1.

11. (canceled)

12. A combination comprising a compound of formula (I), or a pharmaceutically acceptable salt thereof according to claim 1 and at least one further therapeutic agent.

13. (canceled)

14. A pharmaceutical composition comprising a combination comprising a compound of formula (I) or a pharmaceutically acceptable salt thereof according to claim 1 and at least one further therapeutic agent and one or more of pharmaceutically acceptable carriers, diluents and excipients.

15. A combination comprising compound of formula (I) or a pharmaceutically acceptable salt thereof according to claim 1 and at least one further therapeutic agent for use in treating disorders mediated by the target protein.

16. A method of treating disorders mediated by the target protein comprising administering to a human in need thereof a therapeutically effective amount of a combination comprising compound of formula (I) or a pharmaceutically acceptable salt thereof, according to claim 1 and at least one further therapeutic agent.

17. (canceled)

18. A method of degrading the target protein comprising administering to a human in need thereof a therapeutically effective amount of a compound of Formula (I) or a pharmaceutically acceptable salt thereof according to claim 1.

19. A method of treating disorders associated with aberrant kinase activity, wherein the kinase is Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC), said method comprising degrading said kinase.

20. A method of degrading target proteins selected from Adaptor-associated protein kinase 1 (AAK1), Abelson murine leukemia viral oncogene homolog 1 (ABL1), Auorora kinase A (AURKA), Auorora kinase B (AURKB), Bruton's tyrosine kinase (BTK), Cyclin G-associated kinase (GAK), Interleukin-1 receptor-associated kinase 3 (IRAK3), Large tumour suppressor 1 kinase (LATS1), Mitogen-activated protein kinase 9 (MAPK9), Protein kinase AMP-activated alpha-1 (PRKAA1), Focal adhesion kinase (PTK2), Protein tyrosine kinase 2 beta (PTK2B), Ribosomal protein S6 kinase alpha-1 (RPS6KA1), Ribosomal protein S6 kinase alpha-3 (RPS6KA3), Tyrosine-protein kinase Tec (TEC), by constructing Protac compounds or pharmaceutically acceptable salts thereof comprising E3 ligase binding moieties and target protein binding moieties linked directly or via a linking moiety, thus recruiting the target proteins to the E3 ligase allowing ubiquitin transfer from the ligase to the target protein enabling it to be recognized by the proteasome and degraded.

21. (canceled)
